(19) United States
(12) Patent Application Publication
(10) Pub. No.: US 2006/0089636 A1
(43) Pub. Date: Apr. 27, 2006

(54) Ultrasound visualization for transurethral needle ablation

(76) Inventors: Mark A. Christopherson, Shoreview, MN (US); Thomas R. Skwarek, Shoreview, MN (US); Martin T. Gerber, Maple Grove, MN (US)

Correspondence Address: SHUMAKER & SIEFFERT, P. A., 8425 SEASONS PARKWAY, SUITE 105, ST. PAUL, MN, 55125, US

(21) Appl. No.: 10/974,469

(22) Filed: Oct. 27, 2004

(51) Int. Cl. A61B 18/14; A61B 8/12

(52) U.S. Cl. 606/41; 600/439

(57) ABSTRACT

A device and method for transurethral needle ablation (TUNA) of prostate tissue to alleviate BPH provides ultrasound visualization and/or measurement of the urethra, the prostrate, the ablation lesions and/or other pertinent structures. An ultrasound transducer is positioned at the distal tip of the transurethral needle ablation catheter. The ultrasound transducer provides measurements of the target prostrate tissue in each imaging plane before deployment of the ablation needles. The device may also display the imaged tissue for visualization by a physician.

### FIELD OF THE INVENTION

The invention relates generally to prostate treatment and, more particularly, to techniques for transurethral treatment of benign prostatic hypertrophy (BPH).

### BACKGROUND

Benign prostatic hypertrophy or hyperplasia (BPH) is one of the most common medical problems experienced by men over 50 years old. Urinary tract obstruction due to prostatic hyperplasia has been recognized since the earliest days of medicine. Hyperplastic enlargement of the prostate gland often leads to compression of the urethra, resulting in obstruction of the urinary tract and the subsequent development of symptoms including frequent urination, decrease in urinary flow, nocturia, pain, discomfort, and dribbling.

One surgical procedure for treating BPH is transurethral needle ablation (TUNA). The TUNA technique involves transurethral delivery of an electrically conductive needle to the prostate site. The needle penetrates the prostate in a direction generally perpendicular to the urethral wall, and delivers electrical current to ablate prostate tissue. The electrical current heats tissue surrounding the needle tip to destroy prostate cells, and thereby create a lesion within the prostate gland. The destroyed cells may be absorbed by the body, infiltrated with scar tissue or become non-functional.

U.S. Pat. No. 6,551,300 to McGaffigan discloses an example of a transurethral ablation device that deploys a plurality of ablation needles and permits repositioning of the needles within different target sites in the prostate. U.S. Published Patent Application no. 2002/0183740 to Edwards et al. discloses another transurethral ablation device to ablate prostate tissue via electrically conductive needles. U.S. Pat. No. 6,241,702 to Lundquist et al. describes another transurethral ablation needle device. Table 1 below lists documents that disclose devices for transurethral ablation of prostate tissue.

TABLE 1

| Patent Number | Inventors | Title |
|---|---|---|
| 2002/0183740 | Edwards et al. | Medical probe device and method |
| 6,241,702 | Lundquist et al. | Radio Frequency Ablation Device for |
| | | Treatment of the Prostate |
| 6,551,300 | McGaffigan | Device and Method for Delivery of |
| | | Topically Applied Local Anesthetic |
| | | to Wall Forming a Passage in Tissue |

All documents listed in Table 1 above are hereby incorporated by reference herein in their respective entireties. As those of ordinary skill in the art will appreciate readily upon reading the Summary of the Invention, Detailed Description of the Preferred Embodiments and claims set forth below, many of the devices and methods disclosed in the patents of Table 1 may be modified advantageously by using the techniques of the present invention.

### SUMMARY

The present invention is directed to a device and method for transurethral needle ablation (TUNA) of prostate tissue to alleviate BPH that provides ultrasound visualization and/or measurement of the urethra, the prostrate, ablation lesions and/or other pertinent structures. An ultrasound transducer is positioned at the distal tip of a TUNA catheter. The ultrasound transducer provides measurements of the target prostrate tissue in each imaging plane before deployment of the ablation needles. The device may also display the imaged tissue for visualization by a physician.

Various embodiments of the present invention provide solutions to one or more problems existing in the prior art with respect to the ablation of prostate tissue. The problems stem from the fact that prostate sizes and shapes vary over a wide range, and the physician needs to understand the shape and size of the prostrate prior to conducting the TUNA procedure. To gain this understanding, a physician typically performs a standard ultrasound exam prior to the TUNA procedure in order to determine the size and shape of the prostrate. The measured size of the prostate is used to calculate the ablation needle depth. However, the standard pre-procedure ultrasound may not provide the physician with sufficiently detailed information that may be needed during the course of an ablation procedure. For example, the physician typically creates lesions in several planes of the prostate and the dimension of the prostate varies in these different planes. Because detailed size information in all of these different planes may not be available from the standard pre-procedure ultrasound, this could result in needle depths that are too long for certain planes of the prostrate, causing the needles to protrude beyond the prostate lobe upon deployment. Conversely, this can result in under treatment, i.e., lesions having less than optimum size, due to use of a conservative, short needle depth.

Various embodiments of the present invention solve at least one of the foregoing problems. For example, the present invention overcomes at least some of the disadvantages of the foregoing procedures by providing a device and method that provides for ultrasound visualization of the prostate in desired planes of the prostate prior to deployment of the ablation needles. An ultrasound transducer at the tip of the TUNA catheter provides the physician with measurements of the relevant structures, such as the prostate tissue depth (size), in each imaging plane prior to deploying the needles. These measurements can then be used to determine the appropriate needle depth and/or lesion size for the currently imaged plane. This may reduce the need for the standard pre-procedure ultrasound exam and also provides the physician with precise prostate size information at the precise point where the needles will be placed. During the course of the ablation procedure, the ultrasound transducer may also display images and measurements of the lesion itself. The ultrasound image may also allow the physician to see and measure other structures such as the bladder, bladder neck, rectum, urinary sphincter muscle, vascular structures or prostate stones. Visualization of these features may result in a transurethral ablation device and procedure that is safer, faster, more accurate, and more efficient. In addition, the invention provides a transurethral ablation device and procedure that minimizes damage to the urethra, reducing patient pain and recovery time.

Various embodiments of the invention may possess one or more features to solve the aforementioned problems in the existing art. For example, the invention provides a transurethral ablation device and method comprising an ultrasound transducer at the distal tip of the TUNA catheter. In one embodiment, an ultrasound transducer provides an image and/or measurements of the target prostate tissue. The image and/or measurements may be displayed on a graphical user interface. This information may be used the physician to determine an appropriate needle depth and/or lesion size for the currently imaged plane of the prostate.

The invention also provides a transurethral ablation procedure embodied by a method for use of the ablation device described above. The method involves, for example, inserting a distal end of a transurethral needle ablation catheter having an ultrasound transducer at its distal tip into a urethra of a male patient, imaging the target prostate tissue with the ultrasound transducer, displaying the image, deploying at least one ablation needle, and applying ablation energy via the ablation needle.

In comparison to known implementations of transurethral needle ablation, various embodiments of the present invention may provide one or more advantages. An ultrasound transducer at the distal tip of the TUNA catheter allows the physician to accurately visualize and measure the prostate tissue depth (size) in each imaged plane prior to deploying the needles. The physician is thus provided with precise prostate size information in any imaged plane of the prostate. Visualization of the target tissue allows the physician to see the size of the prostate, to use this information to more accurately determine an appropriate needle depth and/or lesion size, and to more safely and accurately place the needles into the prostate at the proper depth and location. Thus, the invention can result in a less complex, more efficient, more convenient and safer procedure. The invention can also result in a procedure in which the risk of damage to the urethra, patient pain and recovery times are minimized, thus promoting patient safety and procedural efficacy.

The ultrasound transducer at the tip of the TUNA catheter may also allow for display and measurement of the lesion itself during the course of the ablation procedure. The physician may thus be provided with lesion size information, and so may more accurately determine when a lesion has reached a desired size. This may result in a more thorough, accurate and effective procedure.

The above summary of the present invention is not intended to describe each embodiment or every embodiment of the present invention or each and every feature of the invention. Advantages and attainments, together with a more complete understanding of the invention, will become apparent and appreciated by referring to the following detailed description and claims taken in conjunction with the accompanying drawings.

The details of one or more embodiments of the invention are set forth in the accompanying drawings and the description below. Other features and advantages of the invention will be apparent from the description and drawings, and from the claims.

### BRIEF DESCRIPTION OF DRAWINGS

FIG. 1 is a schematic diagram illustrating a device for transurethral ablation of prostate tissue in accordance with the invention.

FIG. 2A and FIG. 2B are end and side views, respectively, of the distal end of the device of FIG. 1.

FIG. 3A is a side view of the distal end of a catheter inserted into the urethra of a male patient; FIG. 3B shows an ultrasound transducer array; and FIG. 3B shows an example timing diagram.

FIG. 4 is a flow diagram illustrating a transurethral ablation procedure in accordance with the invention.

FIG. 5 is a flow diagram illustrating a procedure for automatically determining needle depth.

FIG. 6 is a flow diagram illustrating a procedure for automatically determining lesion size.

FIG. 7 is a flow diagram illustrating a procedure for automatically controlling deployment of the ablation needles such that a maximum needle depth is not exceeded.

FIG. 8 is a flow diagram illustrating a procedure for automatically controlling application of ablation energy such that a maximum lesion size is not exceeded.

### DETAILED DESCRIPTION

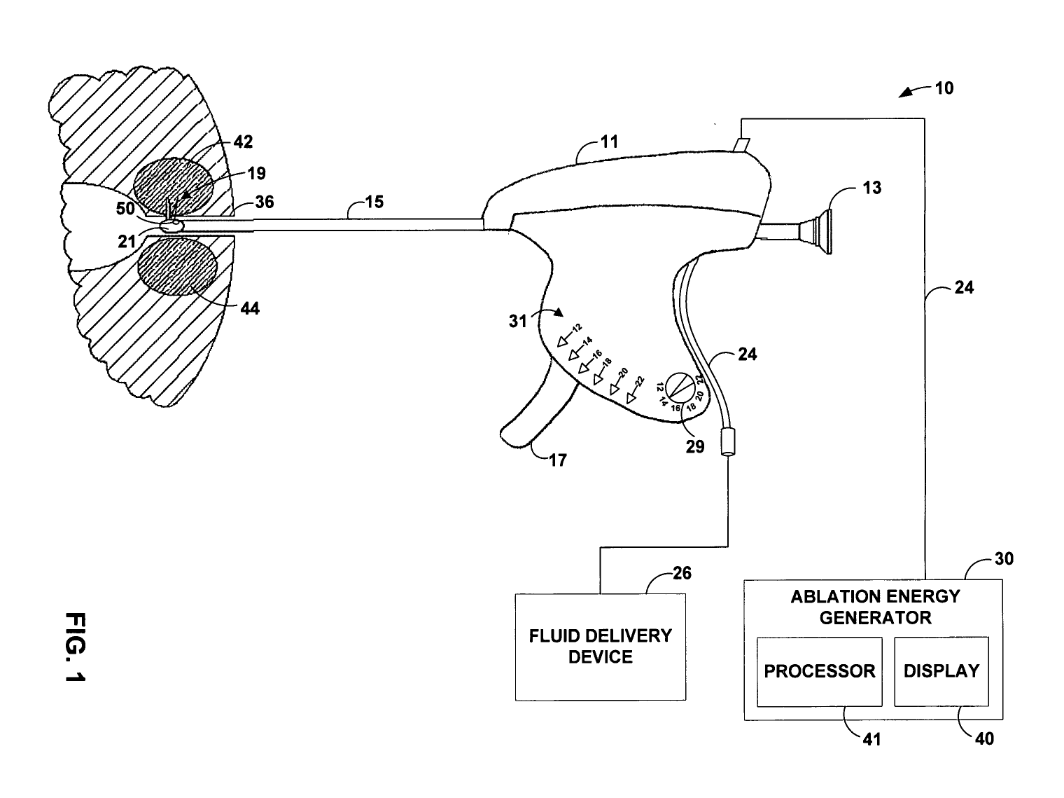

FIG. 1 is a schematic diagram illustrating a device 10 for transurethral needle ablation of prostate tissue. In accordance with the invention, device 10 includes a pair of ablation needles 19A and 19B and an ultrasound transducer 50 at its distal tip 21. Ultrasound transducer 50 provides visualization and/or measurement of the urethra, the prostate tissue, the ablation lesions and/or other pertinent structures. The device may also include other features that will be apparent from this description. Device 10 may generally conform to TUNA devices commercially available from Medtronic, Inc, of Minneapolis, Minn.

As shown in FIG. 1, device 10 includes a handle 11 and a catheter 15 extending from the handle. A trigger-like lever 17 is actuated to advance electrically conductive ablation needles 19A and 19B from a distal end 21 of catheter 15. Device 10 may further include an endoscope viewfinder 13 coupled to an endoscopic transducer (not shown) that extends along the length of catheter 15.

A fluid delivery tube 24 may be coupled to a fluid delivery lumen (not shown) that extends along the length of catheter 15 to deliver fluid to distal end 21. A proximal end of fluid delivery tube 24 is coupled to a fluid delivery device 26 that includes a reservoir containing a fluid and hardware to transmit the fluid to fluid delivery tube 24. For example, fluid delivery device 26 may include a pump, a syringe, or other mechanism to transmit the fluid. Fluid may be delivered to the ablation site for several reasons. For example, delivery of fluid when the catheter 15 is initially inserted helps to clear the view for an endoscope. As another example, delivery of a cooling fluid before, during or after the ablation procedure may provide cooling of the urethra and surrounding tissues to prevent overheating of the urethral wall and any resultant tissue damage. In addition, fluid may be delivered to the target prostate tissue to create a virtual electrode within the target tissue.

An ablation current cable 28 is coupled to an electrical conductor that extends along the length of catheter 15 to needles 19A and 19B. A proximal end of cable 28 is coupled to an ablation energy generator 30. Ablation energy is applied to the prostate tissue via the ablation needles 19A and 19B. The needles 19A and 19B may be unipolar or bipolar. In the unipolar embodiment, ablation energy flows through each needle 19A and 19B while ground pads attached to the patient's skin act as return electrodes. In the bipolar embodiment, ablation energy flows between the needles 19A and 19B and through the surrounding prostate tissue to create a lesion. In another embodiment, a single needle 19 may be used. In that case, the ablation energy may flow between two electrodes carried by the single needle, or between the needle and a ground pad attached to the patient's skin, for example.

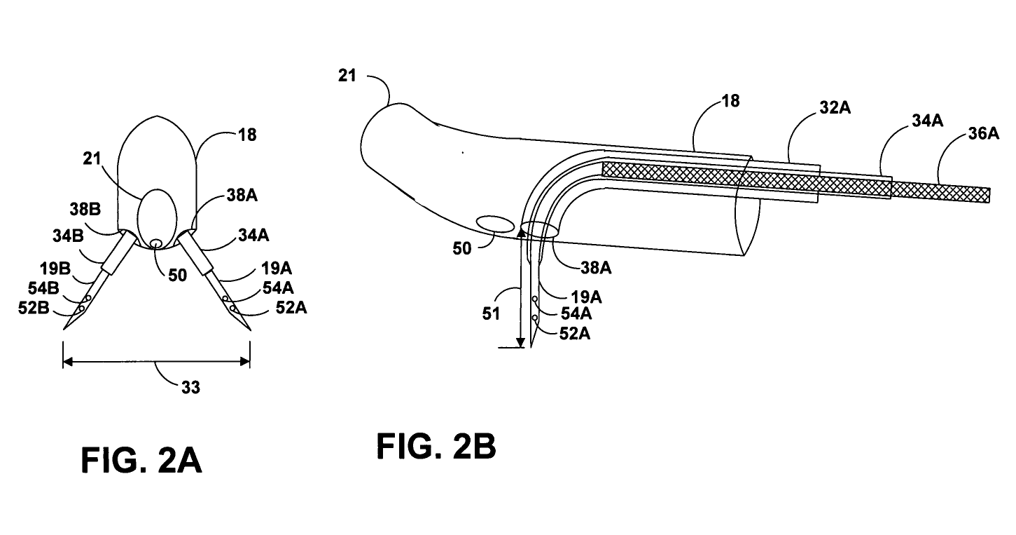

Device 10 may be configured to provide several alternative needle depths. As used herein, “needle depth” refers to the distance that a needle is extended from the distal end 21 of catheter 15. Needle depth is measured from the needle exit port (see FIGS. 2A and 2B) at the distal end 21 of catheter 15 to the tip of the needle 19. In the embodiment shown in FIG. 1, the available needle depths are 12 mm, 14 mm, 16 mm, 18 mm, 20 mm and 24 mm, although may other needle depths could be provided and the invention is not limited in this respect.

Needle depth indicator 31 provides visual feedback to the physician with respect to the needle depth. The physician may use lever 17 to drive needles 19A and 19B through the urethral wall and into prostate lobe 42. To achieve a desired needle depth, the physician may move lever 17 until it is aligned with the markings on needle depth indicator 31 corresponding to the desired needle depth.

Rotary switch 29 also includes indicators corresponding to the needle depths that may be provided by device 10. Rotary switch 29 is set to correspond to the desired needle depth and controls delivery of ablation energy by the ablation energy generator such that the amount of energy delivered to the needle is appropriate for the currently selected needle depth.

The electrical ablation current delivered by needles 19A and 19B may be selected to provide pulsed or sinusoidal waveforms, cutting waves, or blended waveforms that are effective in producing the resistive/ohmic/thermal heating which kills cells within the target tissue site. In addition, the electrical current may include ablation current followed by current sufficient to cauterize blood vessels. The characteristics of the electrical ablation current are selected to achieve significant cell destruction within the target tissue site. The electrical ablation current may comprise radio frequency (RF) current producing power in the range of approximately 5 to 300 watts, and more preferably 5 to 50 watts, and can be applied for approximately 15 seconds to 3 minutes. If electrocautery is also provided via needles 19, then ablation energy generator 30 also may generate electrocautery waveforms.

Ultrasound transducer 50 provides visualization and/or measurement of the urethra, the prostate tissue, the ablation lesions and/or other pertinent structures. Visualization of the target prostate tissue allows the physician to see and measure the size of the prostate to determine an appropriate needle depth, to determine an appropriate lesion size, and/or to position the needles 19A and 19B within the prostate at the proper depth and location. The image from the ultrasound transducer 50 may be acquired and processed via the ablation energy generator 30 and displayed on an associated graphical user interface 40. User interface 40 may be integrated with ablation energy generator, may be a separate, stand alone device, or may be associated with another therapy device. A standard endoscopic viewfinder 13 and camera may also be used in combination with ultrasound transducer 50.

In operation, a physician introduces catheter 15 into urethra 36 of a male patient, and advances the catheter so that distal end 21 is deployed adjacent the prostate. Ultrasound transducer 50 provides visualization and/or measurement of the target prostate tissue for each imaged plane. The transducer may be rotated to obtain views and/or measurements of successive image planes. The ultrasound image and the associated measurements, such as depth, width, length and/or volume of the prostate lobe or any other structures in the imaged plane, may be displayed on user interface 40. In one embodiment, the physician uses this information to properly position the distal end 21 of the catheter 15 relative to the prostate lobes, to determine an appropriate needle depth for the prostate tissue in the currently imaged plane, or to determine an appropriate lesion size for the prostate tissue in the currently imaged plane. In another embodiment, the measurements of the target tissue are analyzed by a processor within ablation energy generator 30 to automatically provide a suggested needle depth or lesion size for the prostate tissue in each imaged plane. Endoscopic viewfinder 13 may also aid in positioning distal end 21 of catheter 15 relative to the prostate lobes.

Distal end 21 is deployed between lateral lobes 42, 44 in the example of FIG. 1. Needles 19 are extended from distal end 21 of catheter 15 to penetrate the urethral wall and one of the prostate lobes 42, 44. In some embodiments, catheter 15 may carry multiple pairs of ablation needles on opposite sides of the catheter to simultaneously access both lobes 42, 44.

Prior to activation of ablation energy generator 30 to deliver ablation current to needles 19, fluid delivery device 26 may be activated to deliver the fluid to the target prostate tissue. The fluid may function to cool the urethral wall in the area of the ablation during the ablation procedure. In another embodiment, fluid delivery device 26 may deliver a fluid that is hyper-echoic directly into the target prostate tissue. Namely, a hyper-echoic fluid of the type that enhances the ultrasound echoes to produce a more accurate or complete ultrasound picture may be delivered directly into the prostate tissue.

In yet another embodiment, fluid delivery device 26 may deliver a fluid that is conductive, such as saline, or a fluid that is loaded with a conductive material. In this manner, the fluid may serve the purpose of creating a virtual electrode to enhance the ablation procedure. A virtual electrode can be substantially larger in volume than the needle tip electrode typically used in RF interstitial ablation procedures and thus can create a larger lesion than can a dry, needle tip electrode. That is, the virtual electrode spreads or conducts the RF current density outward from the RF current source into or onto a larger volume of tissue than is possible with instruments that rely on the use of a dry electrode. In other words, the creation of the virtual electrode enables the current to flow with reduced resistance or impedance throughout a larger volume of tissue, thus spreading the resistive heating created by the current flow through a larger volume of tissue and thereby creating a larger lesion than could otherwise be created with a dry electrode. Use of fluid to create a virtual electrode is described in more detail in copending and commonly assigned U.S. patent application Ser. No. 10/835,193, filed Apr. 29, 2004 to Mark A. Christopherson, et al., entitled “Bipolar Virtual Electrode for Transurethral Needle Ablation”, which is incorporated herein by reference in its entirety.

Either or both of needles 19 or distal end 21 of catheter 21 may include one or more ports for emission of the fluid. The fluid may be sufficiently viscous to provide a controllable flow within catheter 15 and out of distal end 21 of catheter 15. Fluid delivery device 26 may be activated to deliver the fluid before, during and/or after the ablation procedure. For example, the fluid may be delivered before the ablation needles 19A and 19B are activated in order to prepare the tissue in and around prostate gland 42 for delivery of the ablation energy. The fluid may be transmitted to the target tissue site, i.e., the region adjacent prostate lobes 42, 44, by a fluid delivery lumen coupled to one or both of needles 19A, 19B. In particular, either one or both of needles 19A or 19B may be hollow and include one or more fluid delivery ports.

Upon penetration of needles 19A and 19B into prostate lobe 42 and delivery of the fluid, the needles 19A and 19B deliver ablation energy from ablation energy generator 30 to ablate the target prostate tissue within the prostate lobe.

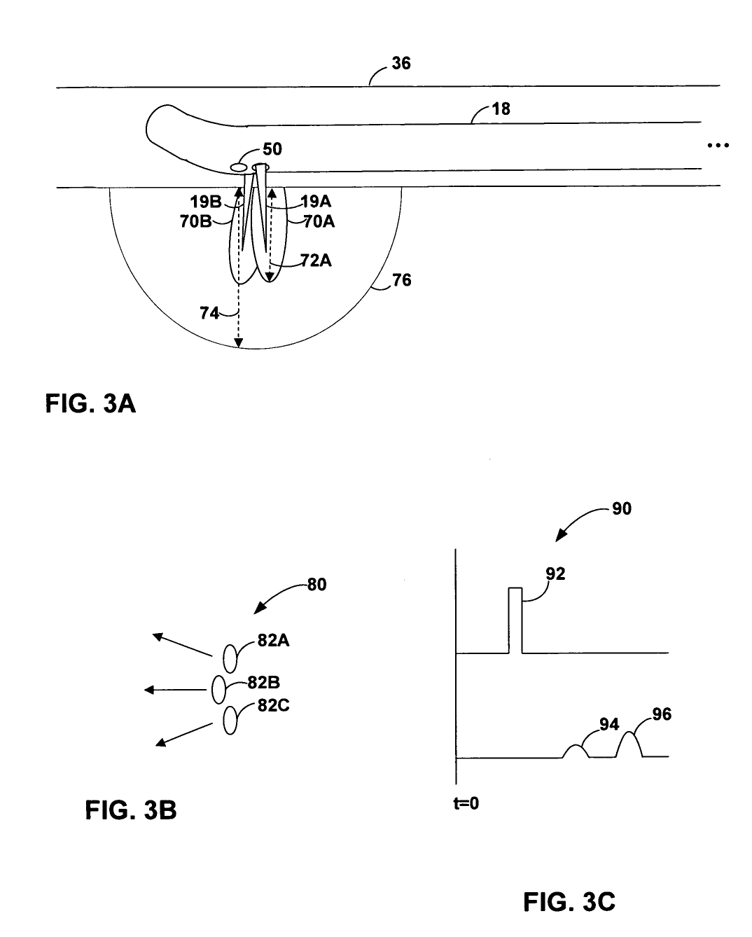

FIG. 2A and FIG. 2B show end and side views, respectively, of the distal end 21 of the device of FIG. 1. Although an exemplary two-needle system is shown in FIGS. 3A and 3B, it shall be understood that single needle systems could also be used and that the invention is not limited in this respect. In addition, three, four or other multiple needle configurations could also be used without departing from the scope of the present invention.

In the embodiment shown in FIGS. 2A and 2B, ultrasound transducer 50 is positioned at the distal end 21 of catheter 15 and is directed toward the direction of needle entry into the prostate tissue. This allows ultrasound transducer 50 to image substantially the same target tissue in which the needles would be deployed. In embodiments where multiple needles simultaneously enter the prostate at different target tissue sites (such as the right and left lateral lobes), multiple ultrasound transducers 50 may be provided, each positioned in the direction of its associated needle entry into the prostate tissue.

Catheter 15 includes guide tubes 32A and 32B (in FIG. 3B, guide tube 32B cannot be seen because it is behind guide tube 32A in this view) extending from the proximal to near the distal end 21 of catheter 15. Needle exit ports 38A and 38B are formed in the wall of the catheter body 15 by the guide tubes 32A and 32B, respectively. Push rods 36A and 36B are connected at their proximal end to a mechanism for deploying the needles 19A and 19B. For example, the push rods 36A and 36B may be operationally connected to the trigger-like lever 17 (see FIG. 1) for deploying and retracting the needles 19A and 19B, respectively, into and out of the prostate tissue. Push rod 36A serves to transfer the mechanical motion of the lever and thus “push” its respective needle 19A out of the exit port 38A of the guide tube 32A and into the prostate tissue. Similarly, push rod 36B serves to transfer the mechanical motion of the lever and thus “push” its respective needle 19B out of the exit port 38B of the guide tube 32B and into the prostate tissue. In one embodiment, the needles 19A and 19B are inserted into the same prostate lobe such that a complete bipolar ablation circuit can be created between the two needles 19A and 19B in a single prostate lobe during the ablation procedure.

Needles 19 may be disposed adjacent one another in a substantially side-by-side relationship as shown in FIG. 2A. In the embodiment of FIG. 2A, needles 19A and 19B exit from the distal end 21 of the catheter 15 at an angle to each other and thus have different insertion points into the prostate tissue, resulting in two different needle “sticks” through the prostate tissue. An insulative sheath 34 surrounds each needle 19 and its corresponding push rod 36. The insulative sheath 34 may extend at least partially into the prostate upon deployment of the needle to avoid undesired ablation of the urethral wall. In the embodiment shown in FIGS. 2A and 2B, each needle 19A and 19B includes fluid delivery ports 52 and 54 for delivery of fluid to the target tissue site. It shall be understood, however, that either one or both of the needles 19 may include fluid delivery ports. Furthermore, it shall be understood that the invention is not limited to the specific type of fluid delivery ports shown in FIGS. 2A and 2B.

Needles 19 may be constructed of a highly flexible, conductive metal such as nickel-titanium alloy, tempered steel, stainless steel, beryllium-copper alloy and the like. Nickel-titanium and similar highly flexible, shaped memory alloys are preferred. Either one or both of needles 19A or 19B may be hollow needles including an internal lumen (not shown in FIGS. 2A and 2B) in fluid communication with fluid delivery ports 52A, 54A, 52B and 54B. In one embodiment, needles 19A and 19B may form opposing polarities for bipolar application of RF ablation current. In this manner, current may be generally confined to the region surrounding needles 19A and 19B and the volume of virtual electrode 48. In another embodiment, ablation needles 19A and 19B are unipolar ablation needles and ground pads attached to the patient's skin may act as return electrodes.

Once deployed from the distal tip 21 of the catheter 15, the needles 19A and 19B are physically spaced apart by the distance indicated by reference numeral 33. The needles 19A and 19B may be spaced apart such that they create a sufficiently large ablation zone. At the same time, the needles may be spaced sufficiently close so that they both penetrate the same prostate lobe. As described above, device 10 may be configured to provide several alternative needle depths. The needle depth is indicated by reference numeral 51. As used herein, “needle depth” refers to the distance that a needle is extended from the distal end 21 of catheter 15. Needle depth 51 is measured from the needle exit port at the distal end 21 of catheter 15 to the tip of the needle 19. In one embodiment, each needle 19A and 19B may have a total depth in the range of approximately 12-22 millimeters, which may be adjustable by the physician as described above with respect to FIG. 1, or which may be fixed in some embodiments. However, many different needle depths could be used and the invention is not limited in this respect. The distance 33 will depend in part upon the depth of the needles and the angle between them. In one embodiment, for example, the distance 33 is in the range of 1±0.5 centimeters.

FIG. 3A shows a side view of catheter 15 having an ultrasound transducer 50 at its distal tip 21 deployed within the urethra 36. Ultrasound transducer 50 uses high frequency sound waves and their echoes to generate a two-dimensional image, or “slice” of the target tissue, namely, the urethra, the prostate lobe, and/or the ablation lesions. Other structures in the imaging plane such as the bladder, bladder neck, rectum, urinary sphincter muscle, vascular structures or prostate stones may also be presented. To produce these images, ultrasound transducer 50 transmits high frequency sound waves into the target tissue. The sound waves travel into the target tissue until they hit a boundary between tissues, such as the edge of a lesion 70A or the edge of prostate lobe 76. At each tissue boundary, some of the individual sound waves are reflected back to ultrasound transducer 50. Some of sound waves that are not reflected are transmitted through the tissue boundary until they hit another tissue boundary and are reflected. The reflected waves are picked up by ultrasound transducer 50 and are relayed to a processor, such as processor 41 (see FIG. 1). Processor 41 controls the amplitude, frequency and duration of the pulses emitted by ultrasound transducer 50. Processor 41 also receives the reflected wave information picked up by ultrasound transducer 50. Processor 41 uses the reflected wave information, including amplitude (intensity) and time of return of each echo, and the speed of sound in the target tissue to calculate the distance to the tissue boundaries. Processor 41 may then use this information to generate and display a two-dimensional image of the target tissue, including the urethra, the prostate tissue, the lesions and/or other structures in the imaging plane of the ultrasound. Measurements corresponding to each of the imaged structures may also be displayed on user interface 40.

In one embodiment, the sound waves produced by ultrasound transducer 50 are in the range of approximately 1 to 15 Megahertz. In another embodiment, ultrasound transducer 50 may provide for multiple frequency imaging of the target tissue at several different frequencies. In addition, ultrasound transducer 50 may include one or more transducer elements. In other words, ultrasound transducer 50 may be a single transducer element or may include a multi-element transducer array. FIG. 3B, for example, shows a multi-element ultrasound transducer 80 having three transducer elements 82A, 82B and 82C. A multi-element transducer array may provide a two or three dimensional view of the target tissue. The ultrasound transducer 50 may therefore vary based on frequency, the number of transducer elements and their individual frequencies, or other ultrasound characteristics, and the invention is not limited in this respect. It shall be understood, therefore, that many different types of ultrasound transducers could be substituted for the specific embodiments shown without departing from the scope of the present invention.

FIG. 3C shows an example timing diagram 90 for a single sound wave 92 emitted by a transducer element. The reflections received by the ultrasound transducer are illustrated by reference numerals 94 and 96. For example, reflection 96 may indicate the edge of a lesion, while reflection 96 may indicate the edge of the prostate lobe. These reflected waves may be analyzed within processor 41 as described above to automatically determine the distances to the tissue boundaries of these structures. Alternatively, a physician may interpret the reflected waves or an image produced based on the waves to determine the distances to the pertinent tissue boundaries.

Referring again to FIG. 3A, in operation, the physician may initially translate and rotate catheter 15 to bring needles 19 into alignment with one of the prostate lobes 76. Ultrasound transducer 50 provides an ultrasound image of the urethra and/or the prostate lobe 76 and may also provide measurements of these features. The image and the corresponding measurements may be displayed on a user interface to aid the physician in proper longitudinal and radial positioning of catheter 15 with respect to the prostate lobe 76.

In addition to aiding the physician in initial positioning of the catheter within the urethra, ultrasound transducer 50 provides a measurement of the depth of the prostate lobe at the current catheter position within the urethra. The measurement of the prostate lobe 74 may be displayed on a user interface before, during and/or after the ablation procedure.

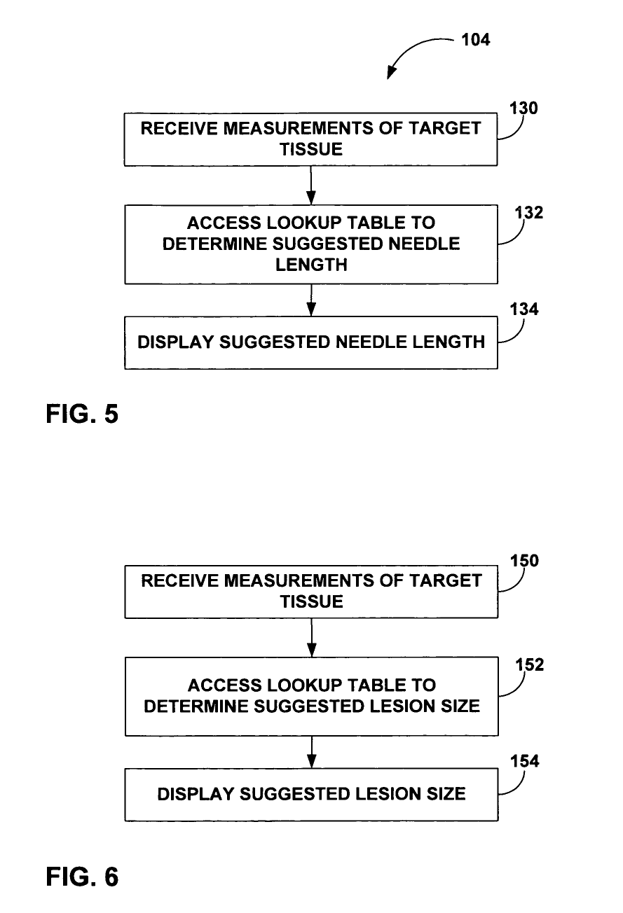

A physician may use the measurement of the prostate lobe 74 to determine an appropriate needle depth for the target tissue in the imaged plane. Alternatively, a processor within the ablation energy generator 30, such as processor 41, may use the measurement of the prostate lobe 74 to automatically determine and suggest an appropriate needle depth for the target tissue in the imaged plane. The automatically determined needle depth may be displayed to the user or may be used to control automatic deployment of the needles to the determined needle depth. Automatic determination of an appropriate needle depth is described in more detail below with respect to FIG. 5.

The measurement of the prostate lobe 74 may also be used to determine an appropriate lesion size for the current catheter position. Alternatively, a processor may use measurement 74 to automatically provide a suggested lesion size, either with respect to an actual, lesion size as measured via ultrasound transducer 50, or in terms of power levels and ablation time necessary to produce such a lesion size. Automatic determination of an appropriate lesion size is described in more detail below with respect to FIG. 6.

Once the proper position, needle depth and/or lesion size have been determined, ablation needles 19A and 19B are inserted into the prostate tissue. For example, a physician may use lever 17 (FIG. 1) to drive needles 19A and 19B through the urethral wall and into prostate lobe 76. Needles 19A and 19B may be inserted together by a single action of the physician or they may be separately controlled.

When needles 19A and 19B are lodged in the prostate lobe 76, the physician may activate fluid delivery device 26 (FIG. 1) to deliver cooling and/or conductive fluid to the target tissue site. The physician next activates ablation energy generator 30 to deliver ablation energy to the target tissue within the prostate lobe and create lesions 70A and 70B via needles 19A and 19B.

Ultrasound transducer 50 may further provide images and/or measurements of each lesion 70A and 70B created by each ablation needle 19A and 19B, respectively. For example, lesion size measurement 72A corresponds to the lesion 70A produced by ablation needle 19A. (Lesion size measurement 72B corresponding to lesion 70B produced by ablation needle 19B is not shown in FIG. 3A). In one embodiment, the lesion sizes 72A and 72B are measured, displayed and updated continuously during the course of the ablation procedure so that the physician has real time information concerning the sizes 72A and 72B of the lesions 70A and 70B, respectively.

The physician may view the displayed lesions 70A and 70B and their associated measurements 72A and/or 72B to determine when the desired lesion size has been reached and/or when the delivery of ablation energy should be stopped. Alternatively, a processor may further use this suggested lesion size to automatically control delivery of ablation energy until the desired lesion size is reached.

After completion of an ablation at the current catheter position, the physician may withdraw the needles, and rotate or otherwise reposition the catheter within the urethra to create additional lesions within the same prostate lobe 76, or to access and ablate another prostate lobe, if desired.

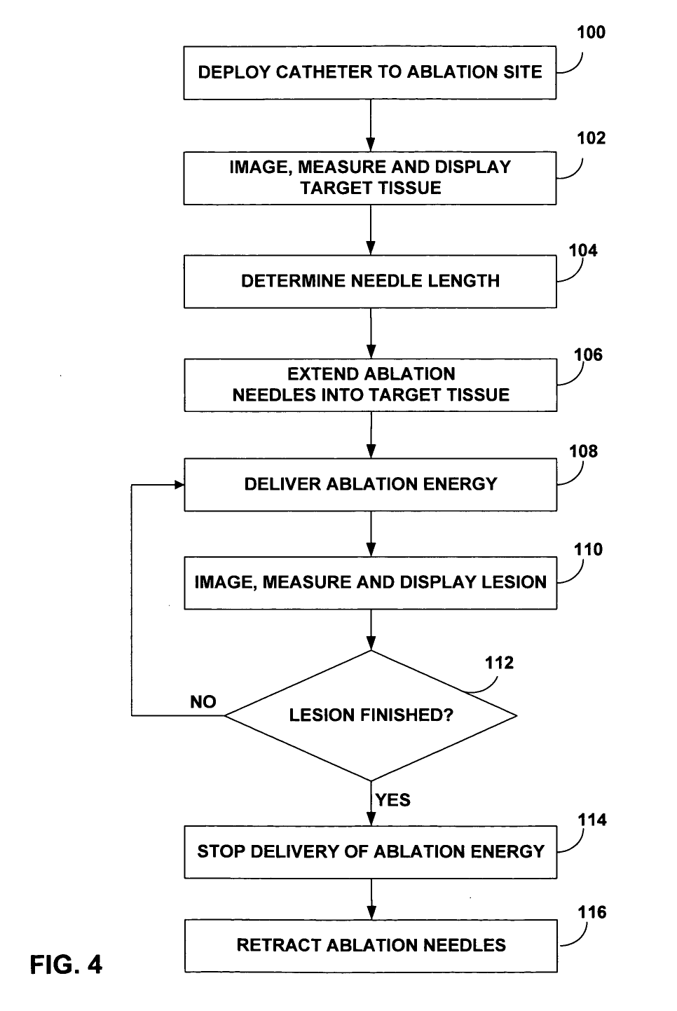

FIG. 4 is a flow diagram illustrating an example embodiment of a transurethral ablation procedure. The procedure involves deploying a catheter to an ablation site (100). The catheter is deployed transurethrally to a position within the urethra corresponding to the target prostate tissue to be ablated. Ultrasound transducer 50 then measures the depth/size of the target tissue and displays the measurements and/or an image of the tissue (102). The physician then uses the displayed measurements and/or the displayed image to determine the appropriate needle depth (104) for the ablation procedure. In another embodiment, a processor analyzes the measured size of the target tissue and automatically suggests an appropriate needle depth for those measurements. This embodiment is described in more detail with respect to FIG. 5. Upon extension of the ablation needles into the target tissue (106), ablation energy is delivered to the ablation needles (108). The ablation energy ablates cells within the target tissue site, creating a lesion within the target prostate tissue. While the ablation energy is applied, ultrasound transducer 50 may continuously or at period intervals image, measure and display the lesion size (110). The physician may use the displayed measurements and image of the lesion with the target prostate tissue to determine when the lesion reaches the desired size. Until the lesion has reached the desired size (112), the physician may continue to apply ablation energy (110) and view the displayed lesion information (110) until the desired lesion size is obtained (112). Once the desired lesion size is obtained, delivery of the ablation energy is stopped (114). The needles may then be retracted back into the catheter (116).

In addition, delivery of ablation energy may be stopped at any point during the process shown in FIG. 4 to allow time for the physician to view the displayed image and/or lesion size information and determine whether the ablation procedure at the current site should continue. The process shown in FIG. 4 may be repeated as many times as necessary at different target tissue sites until all desired lesions are completed.

FIG. 5 is a flow diagram illustrating one example procedure for automatically determining ablation needle depth. Again, “needle depth” refers to the distance that the needles extend from the exit ports 48 of catheter 15. By controlling the amount of extension of the needle from the distal end of the catheter, the depth of penetration of the needle into the target tissue is also controlled. In some embodiments, a processor may be provided to automatically provide a suggested needle depth based on the measurements of the target tissue in the currently imaged plane. The processor may be part of the processor 41 located within ablation energy generator 30 (see FIG. 1) or may be a separate device. For example, in one embodiment, the processor may receive the measurements of the target tissue (130) and access a lookup table having a list of appropriate needle depths corresponding to particular target tissue measurements (132). The device may then display the automatically suggested needle depths on user interface 40 (134) (see FIG. 1).

In one embodiment, the physician may view the suggested needle depths and manually deploy the needles to the suggested depth. In another embodiment, the processor may automatically deploy the needles to the suggested needle depth. The depth of the needle refers to the depth of the needle extended outward from the needle exit ports 38 at the distal end 21 of catheter 15. The processor may first allow the physician to approve of the suggested needle depths before automatically deploying the needles. Or, the device may simply automatically deploy the needles with no input from the physician. Alternatively, the device may allow the physician to override the suggested needle depths and manually deploy the needles to some other physician-determined needle depth. As another example, the automatically determined needle depth may be used as a safety feature. In other words, the automatically determined needle depth may be used to control deployment of the needles. In that embodiment, processor 41 may automatically limit deployment of the needles so as not to exceed an automatically determined maximum needle depth. In one embodiment, processor 41 may cause an audible or visual indication that the maximum needle depth has or is about to be exceeded. In another embodiment, processor 41 may control the lever 17 and/or push rods to prohibit further advancement of the needles. This may help to reduce the danger of advancing the needles through the boundary of the prostate lobe.

FIG. 6 is a flow diagram illustrating one example procedure for automatically determining an appropriate lesion size. In some embodiments, a processor may be provided to automatically provide a suggested lesion size based on the measurements of the target tissue. The processor may be part of the processor 41 located within ablation energy generator 30 (see FIG. 1) or may be a separate device. For example, in one embodiment, the processor may receive the measurements of the target tissue (150) and access a lookup table having a list of appropriate lesion sizes corresponding to particular target tissue measurements (152). The device may then display the automatically suggested lesion size on user interface 40 (154) (see FIG. 1).

In one embodiment, the physician may view the suggested lesion size, perform the ablation and manually stop delivery of ablation energy when the suggested lesion size is achieved. In another embodiment, the processor may automatically stop delivery of ablation energy when the measurements determined using the information from ultrasound transducer 50 indicates that the suggested lesion size has been obtained. The processor may first allow the physician to approve of the suggested lesion size before automatically performing the ablation. Or, the device may simply automatically perform the ablation to the suggested lesion size with no input from the physician. Alternatively, the device may allow the physician to override the suggested lesion size and manually control the ablation until some other physician-determined lesion size has been obtained. As another example, the automatically determined lesion size may be used as a safety feature. In other words, the automatically determined lesion size may be used to control the delivery of ablation energy. In that embodiment, processor 41 may automatically limit delivery of ablation energy so as not to exceed the automatically determined maximum lesion size.

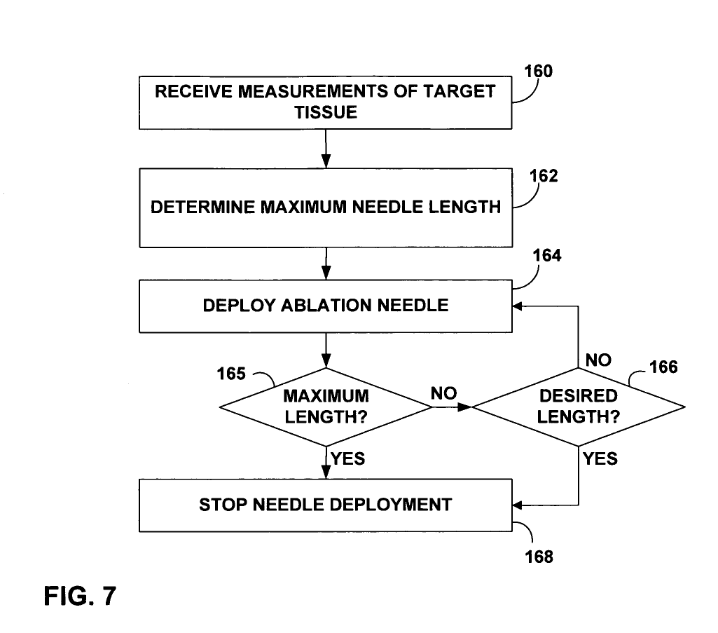

FIG. 7 is a flow diagram illustrating one example procedure for automatically controlling deployment of the ablation needles such that a maximum needle depth is not exceeded. Again, as used herein, needle depth refers to the distance that the needle extends from needle exit port at the distal end 21 of catheter 15. The maximum needle depth may be based in part on the measured sizes of the target tissue or other pertinent structures. To ensure that the maximum needle depth is not exceeded, processor 41 receives the measurements of the target tissue (160). Based on these measurements, processor 41 determines a maximum needle depth for the currently imaged plane of target tissue. The needles are deployed, either automatically or manually (164) and, if the maximum needle depth is reached (165), processor 41 stops deployment of the ablation needles (168). Deployment of the needles may continue, however, until the desired needle depth is reached (166), as long as the maximum needle depth is not exceeded. Once either the maximum needle depth or the desired needle depth is achieved, deployment of the needles is stopped (168) (this can be done either manually by the physician or automatically by processor 41).

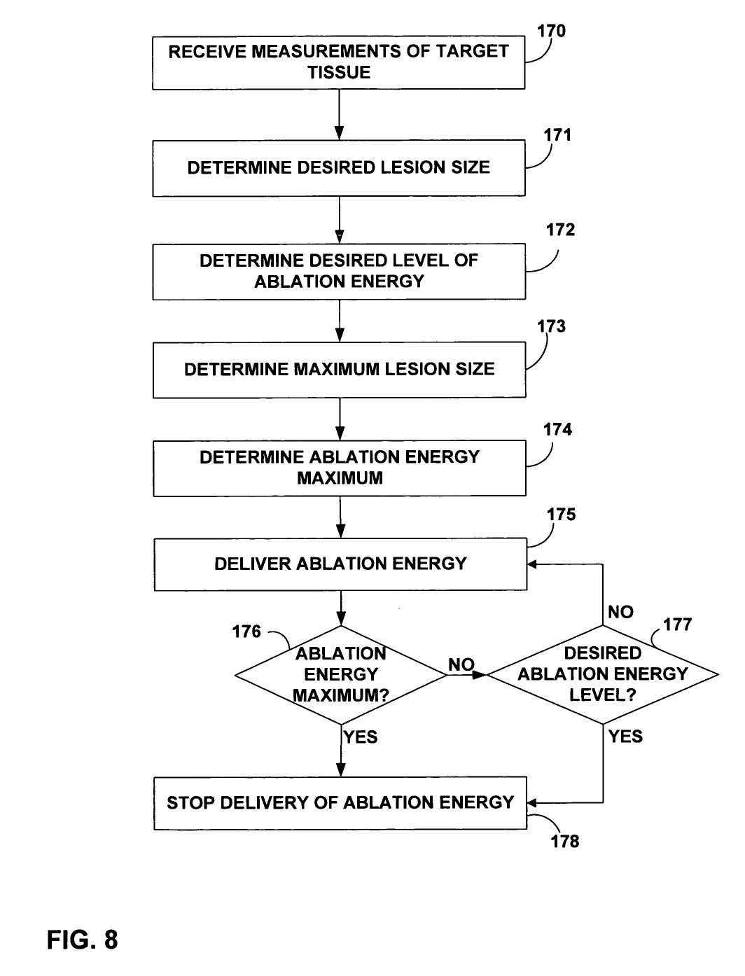

FIG. 8 is a flow diagram illustrating one example procedure for automatically controlling application of ablation energy such that a maximum lesion size is not exceeded. To ensure that the maximum lesion size is not exceeded, processor 41 receives the measurements of the target tissue (170). Based on these measurements, processor 41 determines a desired lesion size (171) and a maximum lesion size for the currently imaged plane of target tissue (173). Processor 41 may then access a lookup table that associates various lesion sizes with the power levels and delivery rates at which to deliver ablation energy to achieve those lesion sizes. In this manner, an associated ablation energy level can be determined that corresponds to the desired lesion size (172) and the maximum lesion size (174). Once delivery of ablation energy begins (175), delivery of ablation energy may be continuously monitored, either automatically or manually and, if the ablation energy maximum is reached (176), processor 41 stops delivery of ablation energy (178). Delivery of ablation energy may continue, however, until the amount of ablation energy required to produce the desired lesion size is reached (177), as long as the ablation energy maximum is not exceeded. Once either the maximum lesion size or the desired lesion size is achieved, delivery of ablation energy is stopped (178). The process outlined in FIG. 8 could also be performed manually by the physician, or may be performed by some combination of manual and automatic control over the process.

Thus, the images and measurements obtained by the ultrasound transducer may be used for several purposes. For example, the ultrasound information may be used to dynamically adjust various parameters during the ablation procedure, including but not limited to controlling needle depth, retracting the needles, varying the power applied to the needles, controlling and adjusting the delivery of fluid, etc.

Although the above description focuses mainly on devices for transurethral ablation of prostate tissue, it shall be understood that the concepts of the present invention may also apply to other medical devices and to other areas of the body. For example, an ultrasound transducer may also be placed in an appropriate position on devices that insert various prosthesis into body tissue. These include devices for the insertion of prosthesis for the treatment of urinary incontinence, transrectally for the treatment of fecal incontinence, or esophageal prosthesis for treatment of gastric reflux or other digestive conditions or any other means of inserting prosthesis via a needle used through the working channel of a cytoscope. Visualization and measurement provided by an ultrasound transducer with those or other devices inserted into channels of the body may allow for more accurate and efficient placement of the device and/or the prosthesis within the body. This approach could also be used for other disease states and corresponding treatments. Examples include cancer, drug delivery for BPH, cancer, or other disease, the administration of cryo, thermo or other type of therapy where an echoic response can be seen.

The visualization and measurement provided by the ultrasound image could also be useful for purposes other than during a transurethral ablation procedure. For example, the images and measurements obtained by the ultrasound transducer could be used to assess post-procedure prostatic swelling, to assess the need for and approximate length of any post-procedure catheterization, to reveal any post-procedure hemorrhaging and to identify blood vessels that may require cauterization, or other post-procedure visualization needs.

The ultrasound images and measurements may also be useful for post-procedure analysis and record keeping. For example, the images and measurements may be printed and or stored and retained to document the procedure. This may provide detailed information concerning exactly where the needles were positioned, the number of ablations performed, and the resulting size of the lesions, resulting in a “map” of the ablated prostate. The ultrasound information may also be kept as part of the patient history for future reference. The information may also be useful for research or other post-procedure analysis of the ablation procedure.

Although the above description focuses mainly on ultrasound imaging, other types of imaging could also be used. For example, magnetic resonance imaging (MRI) devices, thermal imaging devices, or other types of devices for imaging the human body could also be used to image and measure the target prostate tissue.

The invention can provide a number of advantages. In general, the invention provides the physician with more information with which to set up a given ablation procedure. The ultrasound transducer 50 located at the distal tip of the ablation catheter provides measurements and a displayed image of the target tissue in each imaging plane. This results in more accurate determination of the appropriate needle depth and/or for each imaging plane. In addition, visualization and measurement of the lesion size during the ablation procedure provides more control over the resulting lesion size. Because the needle depth may be more precisely determined and the lesions produced may be more precisely sized, the lesion size may be optimized for the particular tissue in each imaged plane. This may reduce the number of times that the needles must be repositioned and redeployed, and may also reduce the total number of lesions which must be created. Visualization of the target tissue before and during the ablation procedure may shorten overall ablation time and may reduce the number of needle “sticks” into the prostate tissue, thus minimizing damage to the urethra, patient pain and recovery time. All of these factors result in a transurethral ablation device and procedure that is faster and more efficient for the physician to perform.

As a further advantage, in some embodiments, the measurements obtained by the ultrasound transducer can be used to automatically determine ablation needle depth, to automatically deploy the needles to the suggested needle depth, and/or to limit deployment of the needles beyond a maximum needle depth, making the procedure less complex, more efficient, and more convenient for the physician and safer and more effective for the patient.

In the claims, means-plus-function clauses are intended to cover the structures described herein as performing the recited function and not only structural equivalents but also equivalent structures. Thus, although a nail and a screw may not be structural equivalents in that a nail employs a cylindrical surface to secure wooden parts together, whereas a screw employs a helical surface, in the environment of fastening wooden parts a nail and a screw are equivalent structures.

Many embodiments of the invention have been described. Various modifications may be made without departing from the scope of the present invention. The preceding specific embodiments are illustrative of the practice of the invention. It is to be understood, therefore, that other expedients known to those skilled in the art or disclosed herein may be employed without departing from the invention or the scope of the claims. For example, the present invention further includes within its scope methods of making and using systems for transurethral ablation, as described herein. These and other embodiments are within the scope of the following claims.

1. A method for performing transurethral needle ablation, the method comprising:
inserting a transurethral needle ablation catheter having an ultrasound transducer positioned at a distal end into a urethra of a male patient;
imaging target tissue with the ultrasound transducer;
displaying the imaged target tissue;
deploying at least one ablation needle into the target tissue; and
delivering ablation energy via the ablation needle.

2. The method of claim 1 further comprising:
determining dimensions of the target tissue based on the imaged target tissue; and
displaying the determined dimensions.

3. The method of claim 1, further comprising determining an ablation needle depth based on the imaged target tissue.

4. The method of claim 3, wherein the ablation needle depth is determined by a physician.

5. The method of claim 4, wherein the physician manually deploys the ablation needle to the determined ablation needle depth.

6. The method of claim 3, wherein the ablation needle depth is automatically determined.

7. The method of claim 6, wherein the ablation needle is automatically deployed to the automatically determined needle depth.

8. The method of claim 3, further comprising
determining a maximum ablation needle depth; and
controlling deployment of the ablation needle such that the maximum ablation needle depth is not exceeded.

9. The method of claim 1, further comprising determining a lesion size based on the imaged target tissue.

10. The method of claim 9, wherein the lesion size is determined by a physician.

11. The method of claim 10, wherein applying ablation energy creates a lesion within the target tissue, and further comprising controlling delivery of ablation energy such that the lesion substantially reaches the determined lesion size.

12. The method of claim 11, wherein the delivery of ablation energy is controlled by a physician.

13. The method of claim 9, wherein the lesion size is automatically determined.

14. The method of claim 13, wherein delivering ablation energy creates a lesion within the target tissue, and further comprising controlling delivery of ablation energy such that the lesion substantially reaches the automatically determined lesion size.

15. The method of claim 14, wherein the delivery of ablation energy is automatically controlled.

16. The method of claim 9, further comprising
determining a maximum lesion size; and
controlling delivery of ablation energy such that the maximum lesion size is not exceeded.

17. The method of claim 1, wherein delivering ablation energy produces a lesion within the target tissue, and further comprising:
imaging the lesion with the ultrasound transducer; and
displaying the imaged lesion.

18. The method of claim 17, wherein imaging the lesion further comprises continuously imaging the lesion during an ablation procedure.

19. The method of claim 9, further comprising determining a level of ablation energy required to produce the determined lesion size.

20. The method of claim 1, wherein the target tissue includes a prostate, and wherein ablation energy includes electrical current selected to kill cells within the prostate.

21. The method of claim 1, wherein delivering ablation energy comprises delivering a radio frequency ablation current via the ablation needle.

22. The method of claim 1, further comprising penetrating a wall of the urethra with the ablation needle, extending the ablation needle into the target tissue, delivering a fluid to the target tissue via the ablation needle, and delivering the ablation energy to the target tissue via the ablation needle.

23. The method of claim 22, wherein the fluid comprises saline.

24. The method of claim 22, wherein the fluid is at least one of electrically conductive or hyper-echoic.

25. A transurethral ablation system comprising:
a transurethral catheter;
an ultrasound transducer positioned at a distal end of the catheter to image target tissue;
at least one ablation needle extendable from the distal end of the catheter to penetrate the target tissue; and
an ablation energy generator to deliver ablation energy to the target tissue via the ablation needle to create a lesion.

26. The system of claim 25, further including a user interface to display the imaged target tissue.

27. The system of claim 25, further comprising a processor to receive imaged target tissue information from the ultrasound transducer.

28. The system of claim 27, wherein the processor determines dimensions of the target tissue based on the imaged target tissue information.

29. The system of claim 28, further including a user interface to display the dimensions of the target tissue.

30. The system of claim 28, wherein the processor determines an ablation needle depth based on the dimensions of the target tissue.

31. The system of claim 30, wherein the processor automatically deploys the ablation needle to the determined ablation needle depth.

32. The system of claim 28, wherein the processor determines a maximum ablation needle depth based on the dimensions of the target tissue.

33. The system of claim 32, wherein the processor controls deployment of the ablation needles such that the maximum ablation needle depth is not exceeded.

34. The system of claim 27, wherein the processor determines a lesion size based on the dimensions of the target tissue.

35. The system of claim 34, wherein the processor automatically controls application of ablation energy to substantially obtain the determined lesion size.

36. The system of claim 27, wherein the processor determines a maximum lesion size based on the dimensions of the target tissue.

37. The system of claim 36, wherein the processor controls application of ablation energy such that the maximum lesion size is not exceeded.

38. The system of claim 25, further including a lookup table memory containing needle depths for each of a plurality of dimensions of target tissue.

39. The system of claim 25, further including a lookup table containing lesion sizes for each of a plurality of dimensions of target tissue.

40. A transurethral ablation system, comprising:
means for imaging target tissue;
at least one ablation needle extendable into the target tissue; and
means for delivering ablation energy to the target tissue via the ablation needle.

41. The system of claim 40, further including means for automatically determining an ablation needle depth based on the imaged target tissue.

42. The system of claim 40, further including means for automatically deploying the ablation needle to the determined needle depth.

43. The system of claim 40, further including means for automatically determining a lesion sized based on the imaged target tissue.

44. The system of claim 43, further including means for automatically controlling application of ablation energy to substantially obtain the determined lesion size.
